(19) United States
(12) Patent Application Publication
(10) Pub. No.: US 2026/0242403 A1
(43) Pub. Date: Aug. 20, 2026

(54) ELECTROCHEMISTRY OF CERTAIN POLYCYCLIC AROMATIC HETEROCYCLES AS APPLIED TO RECHARGEABLE CELLS

(71) Applicant: Michael Anthony Puscaso, Valley Stream, NY (US)

(72) Inventor: Michael Anthony Puscaso, Valley Stream, NY (US)

(21) Appl. No.: 19/054,815

(22) Filed: Feb. 15, 2025

(51) Int. Cl. C07F 9/6568; C07D 335/06; H01M 8/0239; H01M 8/18

(52) U.S. Cl. CPC C07F 9/65683; C07D 335/06; H01M 8/0239; H01M 8/188

(57) ABSTRACT

The molecular structures, solutions, ions, electrodes, compositions, and methods comprising the operation, construction, and use of electrochemical cells containing heterocyclic aromatic compounds as one, if not more, of the primary species participating in the oxidation-reduction reaction(s) that would be characteristic of the cell. Such oxidation-reduction reactions would involve one more transition in the aromaticity of a benzene and/or naphthalene derivative substituted with a heteroatom in the aromatic ring within the cell. The solutions and solvents disclosed herein include a combination of polar, nonpolar, organic, and inorganic solvents. This organic alternative to battery technology does not suppose the use of metals from the environment while also yielding a theoretical voltage that is considerably higher than industry-leading rechargeable cells.

### TECHNICAL FIELD

The present invention relates to electrochemical applications of organic heterocyclic aromatic compounds as applied in oxidation-reduction reactions within rechargeable electrochemical cells.

### BACKGROUND OF THE INVENTION

The current field of rechargeable batteries is far and beyond dominated by the use of metals such as lithium, zinc, manganese, potassium, copper, aluminum, etc. Rechargeable batteries are of particular interest in the energy industry for their applications in virtually all realms of electrical energy storage. As lithium in particular is becoming increasingly scarce and its mining becoming increasingly damaging, innovation is necessary to introduce alternatives that do not rely on metals for production.

The present invention introduces an electrochemical cell that can be synthesized in a laboratory without the input of metals. The structures that will be disclosed herein use the principle of aromaticity as the primary mechanism by which energy is stored in the reactive species.

The compounds have been designed such that electrons are abstracted from or transferred to a single heteroatom within the larger molecule. This ionization therefore converts a ring or group of rings between aromatic and non-aromatic.

The aromatic stabilization energy that is released/absorbed as the reaction progresses is highly significant when compared to the ionization energies of the reactant heteroatoms.

Wherein, the present invention presents a theoretical voltage that exceeds those that are currently available in the consumer market for rechargeable cells.

The present invention is an improvement upon the organic electron storage and transport molecule NAD+ (shown below) occurring naturally in the human body. For the structures disclosed in the present invention (FIGS. 1 & 2), the reactive section of NAD+ has been modified into a bicyclic compound, henceforth referred to as a substituted naphthalene, wherein the single nitrogen atom has been replaced with a single phosphorus or sulfur substituent at the intersection of the two rings.

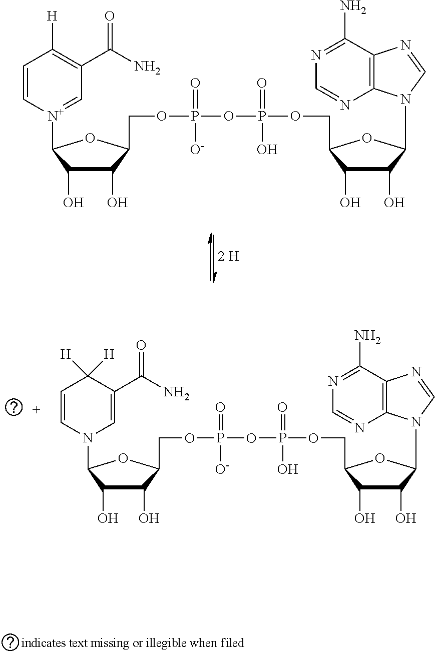

At the cathode is said phosphorus substituted naphthalene, which is being reduced during discharge. At the anode is said sulfur substituted naphthalene, which is being oxidized during discharge. Both naphthalenes will be present in solution with a non-polar organic solvent.

As the cell charging reaction progresses, the previously neutral species will become positively charged in the case of the phosphorus-substituted naphthalene or negatively charged in the case of the sulfur-substituted naphthalene.

This therefore creates a buildup of charge within each half cell which creates a charge imbalance to oppose the intended emf generated by the Gibbs free energy of the reaction. The present invention also introduces a novel mechanism by which current can flow through the battery in order to ensure a balance of charge in each half-cell.

The present invention introduces a gradation of two or more solutions in each half-cell.

The case of two solutions in each cell is only applicable when the chemically active species are in a solid state and therefore reside on the electrodes. More solutions may be necessary if the chemically active species are liquid or aqueous. The present description is made in conjunction with FIG. 3a in which an embodiment with three solutions in each half cell is present.

Each solution is present in its own compartment [15, 16 17, 22, 23, 24] with semi-permeable separators in between each compartment [12]. The gradation of solutions is as follows, in the compartments A and F which contain said graphite electrodes [15, 24], a non-polar organic solvent is present. If the battery is discharged, said substituted naphthalenes are also present in said compartments.

Within the adjacent compartments, B and E [16, 23], is a polar organic solvent that is immiscible with the nonpolar solvent present in compartments A and F. The separator that is present at the interface between said compartments is permeable only at the micro or nano scale therefore preventing mass movements of species between the compartments.

The centermost compartments, C and D [17, 22], contain a polar solvent which demonstrates a considerably larger dipole moment than the polar solvent in compartments B and E and may or may not be organic. The solvent present in compartments C and D is immiscible with the solvent present in compartments B and E. Compartments C and D, while the battery is discharged, contain the majority of the ionic electrolyte that will be used during cell cycling.

The purpose of the gradation is to prevent charged reactive species from each half-cell from ever coming into contact with one another. In addition, said gradation serves to prevent the electrolyte from coming into contact with the highly electronegative or electropositive electrodes.

For example, if there were no gradation, lithium ions in the solution would be reduced by the anode during the charging cycle as opposed to exclusively the sulfur-substituted naphthalene being reduced during charging.

The reaction to form NADH from NAD+ involves reduction of the aromatic ring, therefore destabilizing it, and absorbing the equivalent of the aromatic stabilization energy of the pyridine ring.

The simplified mechanism by which the said reduction is accomplished is through a reaction with a hydride ion at the para position relative to the pyridine ring. This yields an SP3 hybridized carbon at the para position, therefore destabilizing the aromatic pi system of electrons. Subsequently, said ring undergoes bond rearrangement wherein the negative charge from the hydride ion is placed on the formerly positively charged nitrogen atom, therefore yielding a neutrally charged molecule with the proclivity to release 2 electrons at a significant electric potential in order to restore the stabilization energy of the aromatic pi system of pyridine.

The present invention not only provides a significantly more thermodynamically powerful reduction reaction within the disclosed phosphorus-substituted naphthalene but also a novel oxidation reaction within the disclosed sulfur-substituted naphthalene to yield a predicted cell voltage to the order of 7 volts.

### BRIEF DESCRIPTION OF THE DRAWINGS

FIG. 1 shows the two phosphorus-substituted naphthalene compounds included in the present invention. FIG. 1 also demonstrates that said two compounds are an oxidation-reduction pair/couple and indicates that the singular difference between the two compounds is the presence of the electrons in the phosphorus-phosphorus bond.

FIG. 2 shows the two sulfur-substituted naphthalene compounds included in the present invention. FIG. 2 also demonstrates that said two compounds are an oxidation-reduction pair/couple and indicates that the singular difference between the two compounds is the presence of the electrons in the sulfur-sulfur bond.

FIG. 3a shows a subsection of an electrochemical cell. The said cell contains three compartments in each half-cell for a total of six. A solvent is present in each compartment and each compartment is separated from the others by a separator. Current collectors on each side are attached to a series of small, parallel electrodes in the first compartment on each side.

FIG. 3b shows a subsection of an electrochemical cell. The said cell contains two compartments in each half-cell for a total of four. A solvent is present in each compartment and each compartment is separated from the others by a separator. Current collectors on each side are attached to a series of small, parallel electrodes in the first compartment on each side.

FIG. 4 shows a sterically and spatially accurate representation of the phosphorus-substituted naphthalene in the dimer form. The symbol: [symbol] represents a continuation of the bond, meaning that the phosphorus atoms are connected to one another through the same bond. This figure does not accurately illustrate the presence of pi bonds and substituent groups in the said compound, the figure is purely for the illustration of the three-dimensional structure of the said molecule.

FIG. 5 shows a sterically and spatially accurate representation of the sulfur-substituted naphthalene in the dimer form. The symbol: [symbol] represents a continuation of the bond, meaning that the sulfur atoms are connected to one another through the same bond. This figure does not accurately illustrate the presence of pi bonds and substituent groups in the said compound, the figure is purely for the illustration of the three-dimensional structure of the said molecule.

### SUMMARY OF THE INVENTION

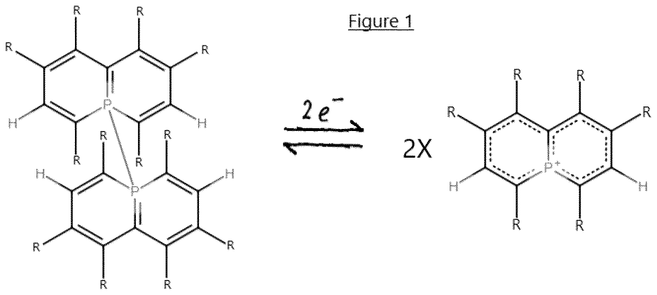

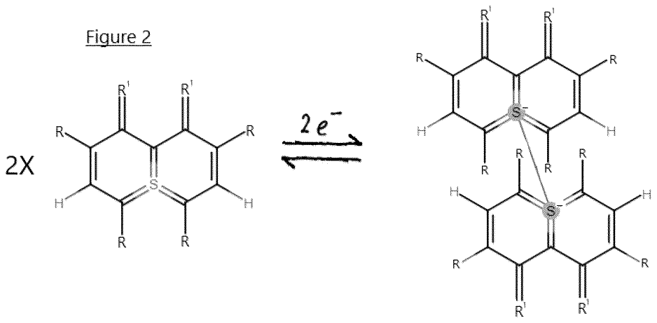

The present invention encompasses the structures enumerated in FIG. 1 and FIG. 2 of the molecular formula (P₂C₁₈H₄R₁₂|[PC₉H₂R₆]⁺) and (SC₉H₂R₄R₂R¹₂|P₂C₁₈H₄R₈R¹₄]²⁻), respectively. Wherein said R groups are independently: Si(alkyl)₃; H; Phenyl; alkyl; thio-alkyl; acyl; nitro; azido; cyano; sulfonyl; ammonium; halogen; haloalkyl; alcohol; or any ether, ester, anhydride or amine thereof. Wherein said R¹ groups are independently: O; C—R₂; or N—R wherein R retains the definition present in the previous sentence.

In the initial/uncharged state, each oxidation-reduction pair of molecules will be present in their unionized states. In the unionized states, the present electrochemical cell is defined to be completely discharged.

In the case of the phosphorus-substituted naphthalene, the molecule that contains the phosphorus-phosphorus bond as seen in FIG. 1 will be henceforth referred to as the phosphorus dimer. The phosphorus dimer represents the discharged state of the phosphorus-substituted naphthalene half-cell.

As the cell charges, the phosphorus-phosphorus bond is broken as an electron is abstracted from each phosphorus atom. As said bond breaks, the product is two bicyclic aromatic naphthalenes. Each of said two naphthalenes will henceforth be referred to as the phosphorus monomer. The phosphorus monomers represent the charged state of the phosphorus-substituted naphthalene half-cell.

In the case of the sulfur-substituted naphthalene, the molecule that contains the sulfur-sulfur bond as seen in FIG. 2 will be henceforth referred to as the sulfur dimer. The sulfur dimer represents the charged state of the sulfur-substituted naphthalene half-cell.

As the cell discharges, the sulfur-sulfur bond is broken as an electron is abstracted from each sulfur atom. As said bond breaks, the product is two bicyclic aromatic naphthalenes. Each of said two naphthalenes will henceforth be referred to as the sulfur monomer. The sulfur monomers represent the discharged state of the sulfur-substituted naphthalene half-cell.

Said compounds in their uncharged states are present in compartments A & F within which a nonpolar organic solvent is present, as referenced in the drawings [15, 24]. As said compounds become ionized as the cell charges, they cannot be solvated in said nonpolar solvent and thus diffuse across the adjacent separator into compartments B and E which contain a polar solvent.

Meanwhile, ions from the electrolyte solution are diffusing across the compartment B/C separator and the D/E separator so as to maintain current flow through the cell and ensure that electric charge is balanced within each respective half-cell.

The reaction of the phosphorus-substituted naphthalene has a predicted half-cell potential of approximately +/− 5.6 volts. The reaction of the sulfur-substituted naphthalene has a predicted half-cell potential of approximately +/− 2.6 volts.

### DESCRIPTION OF THE INVENTION

The present invention encompasses the 4 molecular structures enumerated in FIG. 1 and FIG. 2 and any of their variations described herein. Said four molecular structures follow the molecular formula P₂C₁₈H₄R₁₂ and [PC₉H₂R₆]⁺ depicted in FIG. 1; SC₉H₂R₄R¹₂ and [P₂C₁₈H₄R₈R¹₄]²⁻ depicted in FIG. 2. Wherein said R groups may be any combination of the following: Si(alkyl)₃; H; Phenyl; alkyl; thio-alkyl; acyl; nitro; azido; cyano; sulfonyl; ammonium; halogen; haloalkyl; alcohol; or any ether, ester, anhydride or amine thereof. Wherein said R¹ groups may be any combination of the following: ═O; ═C—R₂; or ═N—R wherein R retains the definition present in the previous sentence.

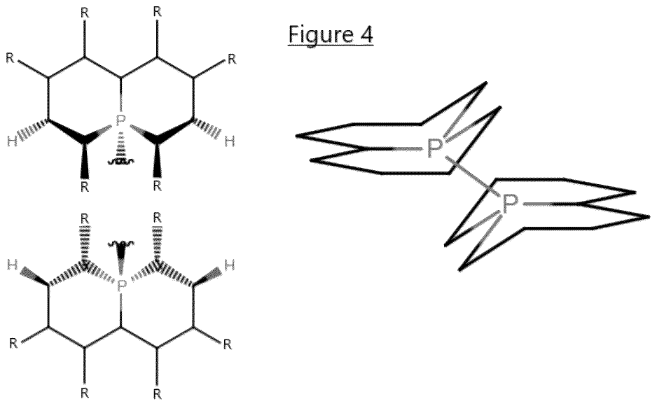

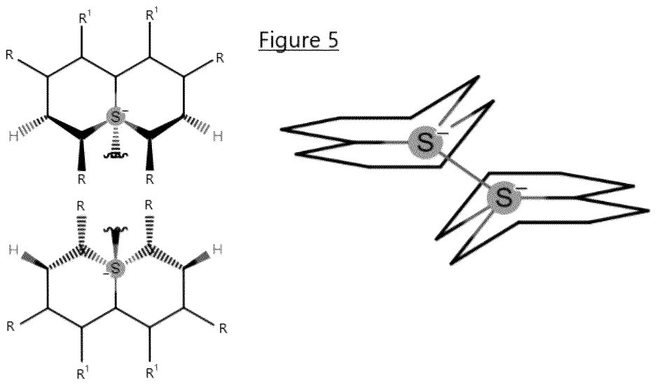

FIGS. 4 and 5 illustrate a sterically and spatially accurate representation of the phosphorus-substituted naphthalene and the sulfur-substituted naphthalene respectively in the dimer forms. Said figures do not accurately illustrate the presence of pi bonds and substituent groups in said compounds, said figures are purely for the illustration of the three-dimensional structure of the molecule.

The bulk of the molecule in the dimer state is approximately planar with the ortho positions of the ring forming an angle of approximately 125 degrees with respect to the rest of the ring. This angle, containing one of the meta positions as its vertex, therefore places the substituent-containing bond of the meta position as co-planar with the phosphorus-phosphorus or sulfur-sulfur bond. Hence only hydrogen can exist at this meta position so as to not encroach upon the opposing meta substituent of the opposing ring.

Other functional groups inserted as R or R¹ groups serve the purpose of sterically protecting the phosphorus and sulfur-substituted naphthalenes when they are in their charged state. In order to ensure against the degradation of the battery's charge and its chemically active species, the charged species must not have the ability to react with other species present in the cell. This is of particular concern when a graphite electrode is used.

The graphite electrode has a high propensity to be oxidized by electrophilic species present in the cell such as the charged phosphorus monomer. Therefore, the electrophilic phosphorus must be protected by sterically prohibiting groups where possible throughout the ring, especially at the ortho position relative to the phosphorus atom. The sulfur-sulfur bond of the charged sulfur dimer must also be similarly protected to prevent it from attacking other species present in the cell.

The steric protection provided by said R groups only protects against the exchange of electrons as a result of collisions. Therein collisions would exchange electrons to form bonds.

Steric hindrance, however, does not prevent the conductance of electrons to or away from species wherein no bonds are being formed because such an interaction does not involve a collision. This is due to the nature of the wave function of electrons.

In addition, the aromatic pi system of electrons, when present, is conductive at the intramolecular level. Therefore, electrons can be conducted from the para position of the phosphorus monomer to the phosphorus atom itself, for example, further increasing the ability of electrons to be exchanged to and from reactant species without a collision.

Given that the exchange of electrons between the graphite electrode and the heterocycles is not possible through a direct collision, this will prevent the degradation of both the electrode and the reactant species during storage when the cell is neither being charged nor discharged. Therein, it is only possible for electrons to be exchanged through conduction over distances at the micro and nanoscale.

Conduction will only occur across sufficient potential, therefore, conduction can only occur when the battery is being either charged or discharged. At the atomic level, the electromotive force supplied across the cell during charging is able to pervade the entire solution at a small distance away from the electrode which provides the necessary activation energy to eject electrons from their bonding orbitals and conduct them through the solution to the electrode. The reverse is true for discharge.

Each oxidation reduction pair described above and depicted in FIG. 1 and FIG. 2 undergoes a change in the molecule's aromaticity as the reaction progresses. Each of the two pairs will start as in their discharged, un-ionized states. In the case of the phosphorus-substituted naphthalene, the discharged state is the dimer and is non-aromatic. In the case of the sulfur-substituted naphthalene, the discharged state is the monomer and is aromatic.

During the charging process, the phosphorus-phosphorus bond in the phosphorus dimer is broken, therefore converting said phosphorous atoms from sp3 hybridized to sp2 hybridized because the bonding electrons are no longer present in the orbital geometry. This transition to sp2 hybridization was the only characteristic preventing the aromaticity of the bicyclic ring which has now become the phosphorus monomer. Therefore, the phosphorus-substituted naphthalene undergoes a transition from non-aromatic to aromatic during the charging reaction in which electrons are abstracted from the said molecule.

During the charging process, a sulfur-sulfur bond is formed between the sulfur atoms of two sulfur monomers, therefore converting said sulfur atoms from sp2 hybridized to sp3 hybridized by adding bonding electrons to the orbital geometry. This transition in the hybridization of the sulfur atoms destabilizes the aromatic system of pi electrons and therefore converts the aromatic sulfur monomer to the non-aromatic sulfur dimer.

### Mechanism

The present invention involves the use of inert electrodes in both half-cells. In the case of a chemically active species which is solid, only two compartments are necessary for said gradation of solvents, otherwise, three or more are needed in each half-cell.

In the case of the solid, the chemically active species would be coated onto the inert electrode in its discharged state and released into the solution in the charged state. Subsequent deposition onto the electrode will occur with each discharge cycle.

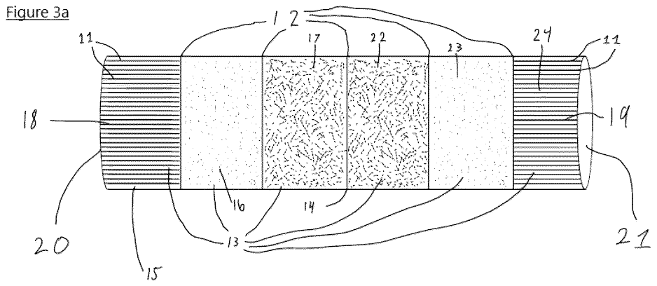

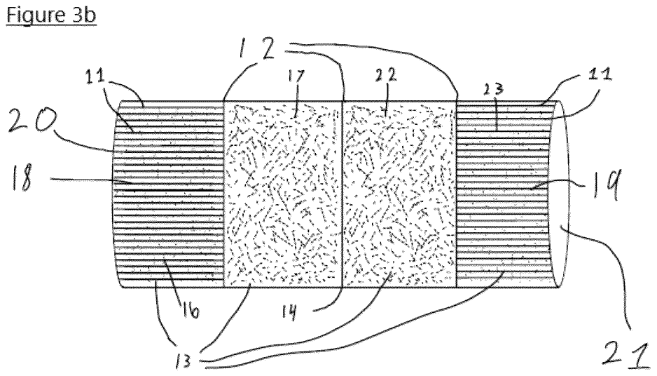

For the purpose of a detailed description of the mechanism, an exemplary embodiment involving graphite electrodes, three compartments [13] in each half cell, and aqueous chemically active species being those disclosed in the present invention will be used in addition to 1,3,5 tri-isopropyl-benzene, trichloroethylene, and water as solvents in said compartments [13]. The three-compartment model can be referenced in FIG. 3a. FIG. 3a does not show a complete cell and it can be interpreted as being a small repeating subsection that is ubiquitous throughout the cell.

1,3,5 tri-isopropyl-benzene will be present as the solvent in compartments A [15] and F [24]. 1,3,5 tri-isopropyl-benzene will serve as the non-polar solvent described in the background and summary of the invention. Trichloroethylene will be present as the solvent in compartments B [16] and E [23]. Trichloroethylene will serve as the polar organic solvent described in the background and summary of the invention. Water will be present as the solvent in compartments C [17] and D [22]. Water will serve as the polar solvent with a significantly higher dipole moment than trichloroethylene, as described in the background and summary of the invention.

In the discharged state, the phosphorus dimer disclosed above will be present in compartment A [15] solvated in 1,3,5 tri-isopropyl-benzene alongside a graphite electrode or series of electrodes as depicted in FIG. 3a [11]. The sulfur monomer disclosed above will be present in compartment F [24] solvated in 1,3,5 tri-isopropyl-benzene alongside a graphite electrode or series of electrodes [11].

Once an electric potential is applied across the cell, this induces an electric field within the cell, between the electrodes. Electrons are not permitted to flow between the compartments because of the separators present at the interface between compartments. Said separators may be made of polyethylene, polypropylene, etc.

During charging, the applied EMF (electro-motive force) will motivate the flow of electrons from the electrode in compartment A[15] to the electrode in compartment F[24]. This EMF is the only force/event that will be capable of exchanging electrons among the reactant species. As stated previously, the reactant heteroatoms, sulfur and phosphorus, are so sterically hindered that any collision with any molecule present in the cell will not be able to gain access to the bonding orbitals of said heteroatoms.

Therefore, the only mechanism by which electrons can be abstracted from said orbitals is through an induced EMF that is powerful enough to provide the necessary activation energy to promote the electrons out of their ground state to higher orbitals and completely liberate them from the atom.

The nature of said electric field is such that no collision with the electrode is necessary. This is because the strength of the induced field is powerful enough to abstract electrons from their orbitals at a distance, though this distance will likely be on the nano-scale. This is possible by how the field repels the positive nuclei and attracts the negative electrons.

In the cell containing the phosphorus dimer, the bonding electrons of phosphorus are the first and likely only electrons to be abstracted from the molecule because phosphorus has both the lowest ionization energy and lowest electronegativity of any atom that would be present in compartment A [15]. Higher ionization energies characterize subsequent ionizations due to same-charge repulsions in addition to destabilization of the aromatic pi system.

Likewise, the sulfur atom will be the first and likely only atom in the cell containing the sulfur monomer to gain an electron. This is because sulfur features a significantly higher electron affinity than any other atom that would be present in compartment F [24].

As electrons are being abstracted from the phosphorus atoms in the dimer, each of the two phosphorus atoms develops a positive charge, the phosphorus-phosphorus bond is broken, and each phosphorus atom transitions from sp3 to sp2 hybridized. Therefore, the naphthalene rings of the now phosphorus monomer have transitioned from non-aromatic to aromatic. The aromatic stabilization energy released as a result of this is used to offset the immense ionization energy absorbed with the formation of the phosphorus cation.

Therefore the net reaction involves the exchange of two electrons for each mole of the reaction. Two electrons are abstracted from the phosphorus-phosphorus bond breaking the dimer into a monomer. Two electrons will likewise be donated to form the sulfur-sulfur bond, uniting the two monomers into a dimer.

Electrons will be donated to the sulfur atoms in each sulfur monomer in compartment F [24] from the graphite electrode(s) [11]. Each of the sulfur atoms will develop a negative charge and therefore they will form a sulfur-sulfur bond thus uniting two sulfur monomers into a sulfur dimer. This is accompanied by each sulfur transitioning from sp2 to sp3 hybridized. This transition destabilizes the pi system of electrons and therefore converts the naphthalene rings from aromatic to non-aromatic. Thus absorbing the aromatic stabilization energy of the formerly aromatic rings.

The formation of a dimer from monomers with an EMF present may result from either a collision between the two monomers or proceed via a radical mechanism. Wherein, each of the two monomers gains an electron to form an intermediary radical and immediately following, two radicals collide to create the bond that characterizes the dimer.

### Thermodynamics

The thermodynamic characteristics of the reaction illustrated in (I) and (II) are variable depending on the reaction mechanism being considered. Wherein the total Gibbs Free Energy values for each reaction remained unchanged, however, the portion of the Gibbs Free Energy that will contribute towards supplying a usable E_{cell} voltage will vary depending on the mechanism.

For the charging reaction concerning the phosphorus-substituted naphthalene, 3 distinct thermodynamic events are used to characterize the Gibbs Free Energy of the reaction. 1) The phosphorus atom itself becomes ionized and develops a positive charge; therein two phosphorus atoms are ionized for each mole of reaction. 2) The phosphorus-phosphorus bond is broken. 3) the aromatic stabilization energy of 2 naphthalene rings is released for each mole of reaction.

In the case of charging the half-cell containing the phosphorus-substituted naphthalene and the case of discharging the half-cell containing the sulfur-substituted naphthalene, a dimer is broken down into two monomers. This is significant because the bond between the respective heteroatoms in the dimer is broken in a single elementary step. This is because said reactions involve no collisions between chemical species. Said two reactions will henceforth be referred to as the “single-path reactions”.

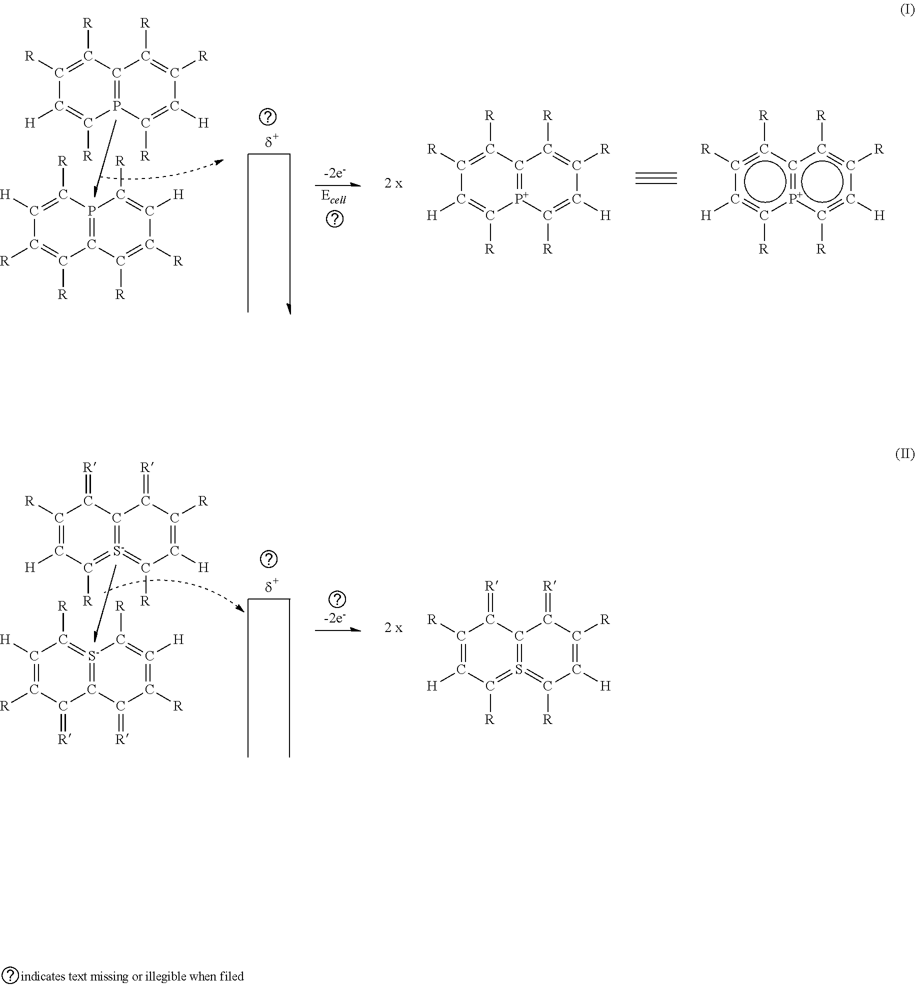

Therefore, in the said reactions, the entirety of the Gibbs free energy produced by the discharge reaction produces a usable voltage, and the entirety of the voltage applied to the cell in the charging reaction is used to store chemical energy in the battery at negligible loss.

However, the remaining two reactions, the charging of the half cell containing the sulfur-substituted naphthalene and the discharge of the half cell containing the phosphorus-substituted naphthalene, are the opposite in that they form a dimer from monomers. These two reactions will henceforth be referred to as the “multi-path reactions” as depicted in (Ill) and (IV).

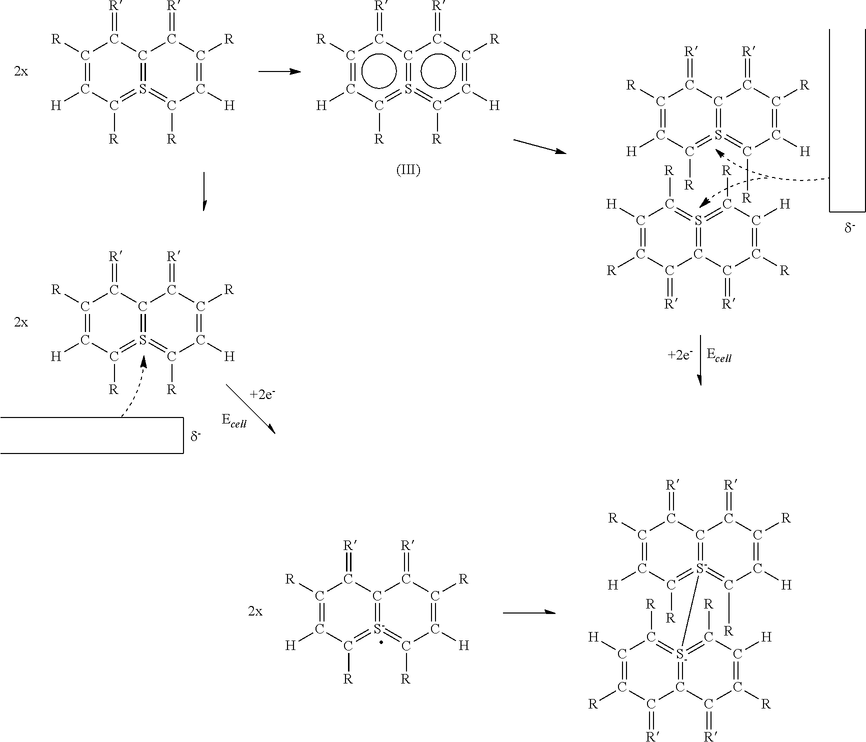

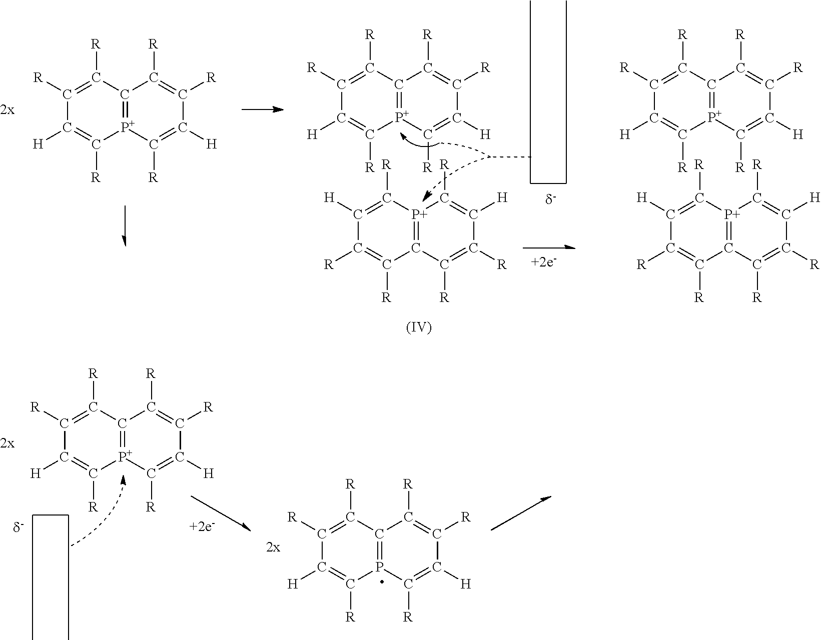

Said two reactions may proceed by multiple mechanisms as shown above. The progression of elementary reaction steps nearest the top of each illustration depicts the mechanism that minimizes losses, such that all Gibbs free energy is used to produce voltage and all voltage is used to store chemical energy in the battery. Said progressions will be henceforth referred to as the “ideal mechanisms”.

The ideal mechanisms involve two of the respective monomers colliding at the proper orientation to react according to the proposed mechanism above. In such a collision, electrons would be donated to the respective heteroatoms, and the inter-heteroatom bond is formed in a single step. Therefore, all the energetic events associated with the Gibbs Free Energy of the reaction occur in concert with the electrode, thus there are negligible losses in cell potential.

However, this mechanism is highly unlikely. The orientation necessary for such a collision is sterically inhibited by the R groups installed at the ortho positions relative to the heteroatom. Though it remains theoretically possible, this mechanism would yield unsatisfactory reaction kinetics which will severely impact the current supplied by the cell and the time necessary to charge it.

The progression of elementary reaction steps nearest the bottom of each illustration depicts the most likely mechanism. However, said mechanism encompasses notable losses. Said progressions will be henceforth referred to as the “un-ideal mechanisms”.

Said un-ideal mechanisms proceed by the formation of a radical as an electron is donated to a species from the electrode as illustrated above. The formation of a radical changes the molecular geometry of the heteroatom from trigonal planar to tetrahedral such that the steric hindrance present in the ideal mechanism is no longer present. The tetrahedral geometry exposes the heteroatom to react with another radical in order to form the resulting dimer.

The formation of a bond from radicals introduces another step to the reaction mechanism which does not occur in concert with the electrode. Since the formation of said bond does not make any contribution to inducing an electric potential through the electrode, the energy released from the formation of said bond will be lost entirely as heat.

The charging reaction in the half-cell containing the sulfur-substituted naphthalene (Ill) can be controlled wherein the ideal mechanism is allowed to be the only mechanism followed. It is important to consider that since the formation of this bond releases energy, this would stipulate that a lower total cell potential is required in the ideal mechanism.

This is because the energy released from bond formation would contribute to the energy absorbed during the ionization of the sulfur atom. Therefore, the reaction may be allowed to proceed exclusively by the ideal mechanism if the supplied charging voltage is controlled to not exceed the voltage necessary to promote the un-ideal mechanism.

The loss of bond enthalpy remains true for the un-ideal discharge reaction of the phosphorus-substituted naphthalene (IV). However, since this reaction occurs only in the discharge cycle, there is no way to control the mechanism by which it proceeds. Therefore it would be logical to conclude that the discharge reaction proceeds mostly by the un-ideal mechanism and therefore the phosphorus-phosphorus bond enthalpy will be entirely lost as heat and the overall cell potential that the battery may supply will be hindered.

### Solutions and Solvents

The present invention utilizes a variety of solvents in each half-cell. This is done such that certain species are soluble in certain compartments [13] while not in others. Each solvent will be immiscible with the other solvents utilized within the cell. Solvents from one compartment are kept as isolated as possible; the effects of surface tension and immiscibility will prevent the mixing of solvents beyond a few molecules.

Between each compartment in the present invention is a separator [12] that is semi-permeable at the molecular level. Said separators will be made from an organic polymer such as polyethylene, polypropylene, etc. Said separators will have pores at the micro and nano scale therefore permitting movement of species at the molecular level. Said separators will act as insulators to prevent the flow of electrons from one electrode to the other.

Each half-cell of the battery will contain said compartments [13] with said solutions and separators. At the interface between the two half cells is a separator [14]. This separator may be exactly the same as those described above [12] such that the same separator is used throughout. It is preferred however, that said separator [14] bear a smaller pore size than the other separators in the cell to further inhibit the movement of species between the two half-cells.

For the purpose of a detailed description of the solutions and solvents present in each compartment of the present invention, an exemplary embodiment involving graphite electrodes, three compartments [13] in each half cell, and aqueous chemically active species being those disclosed in the present invention will be used in addition to 1,3,5 tri-isopropyl-benzene, trichloroethylene, and water as solvents in said compartments [13] and lithium nitrate as the electrolyte. The three-compartment model can be referenced in FIG. 3a.

1,3,5 tri-isopropyl-benzene will be present as the solvent in compartments A [15] and F [24] with the electrodes. 1,3,5 tri-isopropyl-benzene will serve as the non-polar solvent described in the background and summary of the invention. Trichloroethylene will be present as the solvent in compartments B [16] and E [23]. Trichloroethylene will serve as the polar organic solvent described in the background and summary of the invention. Water will be present as the solvent in compartments C [17] and D [22]. Water will serve as the polar solvent with a significantly higher dipole moment than trichloroethylene, as described in the background and summary of the present invention.

In the discharged state of the battery, the chemically active species will be in their discharged states and will be nearly entirely present in compartments A [15] and F [24] respectively with the respective solvent. The phosphorus-substituted naphthalene will be contained in compartment A while the sulfur-substituted naphthalene will be contained in compartment F. In the adjacent compartments [16][23], only trichloroethylene will be present with possibly a very small amount of precipitate which will be discussed subsequently. Within the central compartments C [17] and D [22], water will be present in addition to the electrolyte that will be used in the operation of the battery.

This system accomplishes the following as will be described herein: 1) it prevents reactive substituted naphthalenes from coming into contact with one another inside the cell. 2) it prevents ions from the electrolyte from coming into contact with the electrodes which will reduce or oxidize said ions. 3) it allows the movement of charge and electric current through the cell as ions diffuse through their respective separators.

As the cell charges, an electric field is induced within the cell. The direction of the said field is from the phosphorus species-containing half-cell to the sulfur species-containing half-cell. Therefore, in order to maintain current flow through the battery, ions must be able to move according to this electric field.

As the phosphorus-substituted naphthalene is ionized at the electrode in compartment A [15], the positive ions are no longer soluble in the 1,3,5 tri-isopropyl-benzene solvent and the positive ions are repelled by the electron-deficient electrode. Therefore, the charged species is forced to diffuse across the adjacent separator into compartment B [16] where it encounters a polar organic solvent and may be properly solvated.

Simultaneously, negatively charged nitrate ions from the electrolyte solution in compartments C [17] and D [22] are being subjected to the said electric field and are therefore being forced toward the electrode in compartment A [15]. However, the ions will be insoluble in the polar organic solvent present in compartment A [15], therefore, they will not go further than compartment B [16].

Since trichloroethylene has little polarity relative to water, it will likely only be able to solvate the ions entering compartment B [16] marginally. Therefore, the majority of the positively charged naphthalenes and nitrates will meet in compartment B [16] and precipitate out of the solution.

The substituted naphthalenes will only be slightly soluble in water and will therefore likely precipitate out in compartment C [17], or reside there in solution. It is unlikely that the charged naphthalenes will make it to compartment C [17] because of how slowly they will move through the solvents during charging and because doing so would create a charge imbalance in compartment C [17] due to most, if not all, of the nitrate diffusing to compartment B [16].

A similar mechanism can be observed simultaneously in the half-cell containing the sulfur-substituted naphthalene. The negatively charged sulfur dimer produced at the electrode in compartment F [24] is no longer soluble in the nonpolar 1,3,5 tri-isopropyl-benzene. The electric field will force the said ions into compartment E [23] where said ions encounter the polar trichloroethylene and may be properly solvated.

Simultaneously, lithium ions from the electrolyte solution in compartments C [17] and D [22] will be subject to the electric field and will therefore be forced toward compartment E without being allowed to travel beyond compartment E [23] to compartment F [24].

A similar precipitation reaction to that happening in compartment B [16] will also be happening in compartment E [23] where the negatively charged sulfur-substituted naphthalene precipitates out of solution with the positive lithium ions. The sulfur-substituted naphthalene will likely not have enough time to diffuse across compartment E [23] into compartment D [22] during the charging cycle.

In summary, during charging, positive charges are created in compartment A [15] which then diffuse to compartment B [16] and marginally to compartment C [17]. Meanwhile, negatively charged ions in the electrolyte diffuse from compartments C [17] and D [22] to compartment B [16]. Negative charges are created in compartment F [24] which then diffuse to compartment E [23] and marginally to compartment D [22]. Meanwhile, positively charged ions in the electrolyte diffuse from compartments C [17] and D [22] to compartment E [23].

Wherein, a flow of charge is now created throughout every compartment in the cell in accordance with the induced electric field across the cell. Therefore, the flow of electric current is maintained throughout the cell and no charge imbalance is created during cell operation.

1. Compounds of the formulae (1) and (II), 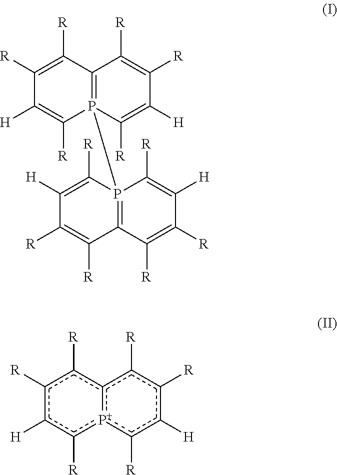
wherein: R is independently selected from the group consisting of Si(alkyl)₃; H; Phenyl; alkyl; thio-alkyl; acyl; nitro; azido; cyano; sulfonyl; ammonium; halogen; haloalkyl; alcohol; or any ether, ester, anhydride or amine thereof.

2. Compounds of the formulae (Ill) and (IV), 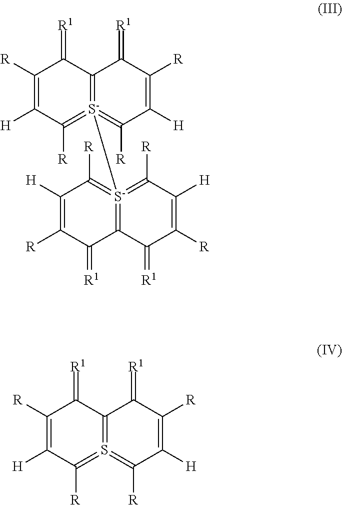
wherein: R groups present in (Ill) and (IV) are independently Si(alkyl)₃; H; Phenyl; alkyl; thio-alkyl; acyl; nitro; azido; cyano; sulfonyl; ammonium; halogen; haloalkyl; alcohol; or any ether, ester, anhydride or amine thereof, R¹ groups present in (Ill) and (IV) are independently O; C—R₂; or N—R, Where R retains the definition provided above.

3. The electrochemical cell comprising two half-cells, wherein each half-cell contains two or more compartments, and each compartment within a half-cell contains a different solvent; a half-cell may share the second compartment with the second half-cell such that there may be 3 compartments total in the complete cell.

4. An electrochemical cell of claim 3 wherein, each of said compartments is separated from other compartments within the cell by a semi-permeable separator.

5. An electrochemical cell of claim 3 wherein, each semi-permeable separator is constructed from organic polymers such as polyethylene, polypropylene, polyesters tetrafluoroethylene, and polyvinyl chloride.

6. An electrochemical cell of claim 3 wherein, each of said solvents present in said compartments exhibit a gradation of polarities as one moves from one end of a half-cell to the other.
